(19) United States
(12) Patent Application Publication
(10) Pub. No.: US 2021/0386757 A1
(43) Pub. Date: Dec. 16, 2021

(54) Methods of Treating Menopausal Symptoms Using Low Dose Progesterone

(71) Applicant: Sarah S. Stadler, Charlottesville, VA (US)

(72) Inventor: Sarah S. Stadler, Charlottesville, VA (US)

(21) Appl. No.: 17/286,184

(22) PCT Filed: Oct. 16, 2019
(86) PCT No.: PCT/US19/56595

§ 371 Date: Apr. 16, 2021

Related U.S. Application Data
(60) Provisional application No. 62/746,753, filed on Oct. 17, 2018.

(51) Int. Cl. A61K 31/57; A61P 15/12

(52) U.S. Cl. CPC A61K 31/57; A61P 15/12

(57) ABSTRACT

This disclosure describes studies undertaken to determine the effectiveness of administration of low dose progesterone monotherapy in alleviating symptoms associated with menopause transition. It is apparent based on initial results that treatment with a low dose progesterone agent alleviates symptoms in many women transitioning into menopause. Progesterone at 25 mg once or twice per day can improve the quality of life for the woman in the early and late phases of menopause without side effects and no risks. Accordingly, this disclosure describes methods for treating one or more symptoms of menopause in a subject. The methods can include administering to a subject who would benefit from such treatment (e.g., a perimenopausal female) a low therapeutically effective amount of a progesterone agent to treat the subject for the one or more symptoms of menopause.

### CROSS REFERENCE

This application claims the benefit of U.S. Provisional Application No. 62/746,753, filed Oct. 17, 2018, which is incorporated by reference herein in its entirety.

### INTRODUCTION

With the onset of menopause in women, symptoms occur owing to altered hormone production. Menopause transition is the time period before and shortly after the cessation of the menstrual cycle. This time period results in a number of symptoms for many women to include; mood changes, hot flushes, breast tenderness, sleep problems, fatigue, and exhaustion. Common treatment options offered to women include; selective serotonin reuptake inhibitors, oral contraceptive pills, high dose progesterone, and hormone replacement therapy. The benefits and risks of hormone replacement therapy with estrogen and progesterone have been well documented. Although hormone replacement therapy (HRT) with estrogens or an estrogen/progestin combination are commonly used for the above symptoms, these options often have side effects and may be ineffective.

One of the risks of estrogen replacement therapy is an increased risk of endometrial carcinoma. The use of simultaneous treatment with estrogen and a progestin mitigates these risks. Progestins suppress the stimulating effect on the endometrium caused by estrogens. Combination therapy (estrogen/progestin treatments) can lead to an increase in breast cancer risk. The search for alternative therapies leads to a combination of an antioestrogen with an antiprogestin and the use of a combination of estrogens and antiprogestins in a sequential manner. For numerous antiprogestins, antiproliferative activity in vitro and antitumor effect in animal models has been reported for compounds such as RU 486, Onapristone, and ZK 230 211.

Consequently, despite the availability of hormone replacement therapy, there continues to be a need for more effective treatment options for the symptoms associated with the menopause transition state.

### SUMMARY

Menopause transition is the time period before and shortly after the cessation of the menstrual cycle. This time period results in a number of symptoms for many women to include; mood changes, hot flushes, breast tenderness, sleep problems, fatigue, and exhaustion. Common treatment options include selective serotonin reuptake inhibitors, oral contraceptive pills, high dose progesterone, and hormone replacement therapy. These options often have side effects and may be ineffective. Avoiding the use of estrogen and administering medication at a lower dose would reduce the risks associated with hormone replacement therapy. It was considered that if symptoms can be alleviated with the use of only one agent, micronized progesterone, at a lower dose, then it is reasonable to assume that risks with medication use would be a safer alternative. In addition, such treatment should have a preventive effect on the occurrence of breast cancer.

This disclosure describes studies undertaken to determine the effectiveness of administration of low dose progesterone monotherapy in alleviating symptoms associated with menopause transition. It is apparent based on initial results that treatment with a low dose progesterone alleviates symptoms in many women transitioning into menopause. Progesterone at 25 mg once or twice per day can improve the quality of life for the woman in the early and late phases of perimenopause without significant side effects and risks.

Accordingly, this disclosure describes methods for treating one or more symptoms of the menopause transition in a subject. The methods can include administering to a subject who would benefit from such treatment a low therapeutically effective amount of a progesterone agent to treat the subject for the one or more symptoms of menopause. The subject can be a female subject in the menopause transition state.

### DETAILED DESCRIPTION

### Methods of Treating Menopausal Symptoms

As summarized above, this disclosure provides methods for treating or preventing symptoms associated with the menopause transition state where there is administered to a subject in need of such treatment a low therapeutically effective amount of a progesterone agent. The subject methods provide for administration of a particular low dosage regimen of the progesterone agent that is effective to at least ameliorate the one or more menopausal symptoms in the subject. In some cases, the subject methods provide for administration of the progesterone agent as a monotherapy for hormone replacement, i.e., administered in the absence of an additional active agent such as an estrogen agent.

The subject in need of such treatment according to the subject methods can be a female in the menopause transition. The time period before and after the cessation of menstruation is referred to as menopause transition. In some case, women enter this stage 8 to 10 years ahead of menopause. In certain cases, a woman can begin the transition into menopause during her 30s or 40s. Most women who have symptoms related to hormonal changes present in the menopause transition time period. The corpus luteum is a structure in the female ovary that occurs with ovulation. The corpus luteum is responsible for producing relatively high levels of progesterone. During the menopause transition time, ovulation becomes less frequent and often ceases. Lack of an adequate corpus luteum cyst results in a decline in progesterone production. Many women who suffer from symptoms during this time period likely produce less progesterone in relationship to estrogen. The biosynthesis of estrogens differs between the premenopausal and postmenopausal woman. In the premenopausal woman, estrogen is primarily made in the ovary. In the postmenopausal women, estrogen is synthesized in peripheral sites. The main source of estrogen in the obese postmenopausal woman is the fat tissue. Fat tissue produces excess amounts of estrogen which can put a woman at risk of breast, endometrial, ovarian, and other cancers.

The menopause transition time, referred to as perimenopause, includes both the early and late perimenopausal group. Late perimenopause was defined as menstruation in the previous/last 2-12 months, but not in the previous/last 2 months. Early perimenopause was defined as increasing irregularity of menses without skipping periods. Common symptoms during this time are mood swings, hot flushes, breast tenderness, sleep disturbances, and fatigue.

The menopausal symptoms which may be treated according to the methods of this disclosure include, but are not limited to, those described by Greene, J. G. and Cooke, D. J. (1980) British Journal of Psychiatry Volume 136, 486-491. In some cases, the menopausal symptoms treated or prevented according to the present disclosure include hot sweats and night time sweats, hot flashes, sleep disturbances, fatigue, breast tenderness and emotional lability. Having said this, the method of the disclosure is also applicable to the treatment and/or prevention of any convenient symptoms of menopause as previously described.

In some embodiments, postmenopausal women can be treated with the methods of the disclosure.

Aspects of the disclosure include methods of hormone replacement therapy in a subject. The hormone replacement methods can include administering to a woman a low therapeutically effective amount of progesterone as a monotherapy to treat the subject for progesterone deficiency. In some embodiments, the method further includes identifying the woman as being in need of hormone replacement therapy prior to administering the progesterone to the woman.

In some embodiments, a woman subject can be identified as being in need of hormone replacement therapy (using standard criteria, as described, for example, by the American College of Physicians Guidelines (incorporated herein by reference)) prior to treatment of the woman according to the methods of the disclosure. A variety of therapeutic regimens are suitable for use in the disclosure, and practitioners of ordinary skill in the art can readily optimize a particular regimen for a particular woman by monitoring the woman for signs and symptoms of hormone deficiency, and increasing or decreasing the dosage and/or frequency of treatment as desired.

Treating progesterone deficiency in the patient transitioning into menopause according to the subject methods may reduce risks of the side effects that can occur with conventional therapies involving progesterone drugs or HRT involving estrogens and/or progesterones. See e.g., Siddique et al. (“Genotoxic potential of medroxyprogesterone acetate in cultured human peripheral blood lymphocytes” Life Sciences 80 (2006) 212-218).

In certain embodiments, the subject methods utilizing a low therapeutically effective amount of a progesterone agent can provide for at least amelioration of one or more symptoms of menopause (e.g., as described herein). In certain embodiments, the one or more symptoms of menopause are selected from alteration in mood, hot flashes/flushes, sleep disturbances, fatigue, physical and/or mental exhaustion, breast tenderness and emotional lability. In some embodiments, the method results in a reduction in one or more symptoms as measured on the Menopause Rating Scale (MRS). In certain embodiments, the one or more symptoms include alteration of mood as defined as depressive mood, irritability, or anxiety on the Menopause Rating Scale (MRS).

In certain embodiments, the one or more symptoms include hot flushes, sleep problems, depressive mood, irritability, anxiety, and physical/mental exhaustion.

In certain embodiments, the subject methods utilizing a low therapeutically effective amount of a progesterone agent can provide for a reduced occurrence of, or amelioration or, one or more side effects associated with conventional progesterone therapy, including but not limited to, headache, breast tenderness or pain, upset stomach, vomiting, diarrhea, constipation, tiredness, muscle, joint, or bone pain. mood swings, irritability, excessive worrying, runny nose, sneezing, cough, vaginal discharge, problems urinating, breast lumps, migraine headache, severe dizziness or faintness, slow or difficult speech, weakness or numbness of an arm or leg, lack of coordination or loss of balance, shortness of breath, fast heartbeat, sharp chest pain, coughing up blood, leg swelling or pain, loss of vision or blurred vision, bulging eyes, double vision, unexpected vaginal bleeding, shaking hands that you cannot control, seizures, stomach pain or swelling, depression, hives, skin rash, itching, difficulty breathing or swallowing, swelling of the face, throat, tongue, lips, eyes, hands, feet, ankles, or lower legs and hoarseness.

In certain instances, the subject methods can provide for treatment without increased risk of cancer, e.g., as can be associated with conventional HRT. In certain embodiments, the subject methods utilizing a low therapeutically effective amount of a progesterone agent can provide for effective treatment without increased risk of cancer (relative to no therapy), or with a reduced risk or occurrence of cancer as compared to conventional therapies that involve administration of a high dosage regimen of a progesterone agent. In some cases, the cancer of interest is breast, endothelial or ovarian cancer. Treating progesterone deficiency in a patient according to the subject methods may provide treatment without risk of the above-mentioned cancers.

The term “progesterone agent” refers to an agent, natural or synthetic, that effects some or all of the biological changes produced by progesterone or progestin, which is a major female steroid hormone of the corpus luteum. For example, a progestin can induce secretory changes in the endometrium that is natural progesterone. In some embodiments, a progesterone agent is a selective progestin receptor modulator (SPRM) capable of acting as a progesterone receptor agonist. Progesterone agents of interest, include but are not limited to, progesterone (C₂₁H₃₀O₂), medroxyprogesterone acetate (C₂₄H₃₄O₄), d-norgestrel (C₂₁H₂₈O₂), norethindrone (C₂₀H₂₆O₂), 17-hydroxy progesterone derivatives, 19-nor testosterone derivatives, 19-nor-progesterone derivatives, norethindrone, norethindrone acetate, norethynodrel, norgestrel, norgestimate, ethynodiol diacetate, allylestrenol, lynoestrenol, fuingestanol acetate, medrogestone, norgestrienone, dimethiderome, ethisterone, cyproterone levo-norgestrel, dl-norgestrel, cyproterone acetate, gestodene, desogestrol, phytoprogestins, dydrogesterone, ethynodiol diacetate, medroxyprogesterone acetate, megestrol acetate, animal-derived progestins, and metabolic derivatives of animal-derived progestin. In certain embodiments, the progesterone agent is selected from RU486, CDB2914, 19-nor-progesterone derivatives, 19-nor-testosterone derivatives, 6-aryl-1,2-dihydro-2,2,4-trimethylquinoline derivatives, 5-aryl-1,2-dihydro-5H-chromeno [3,4-f] quinoline derivatives, 5-alkyl 1,2-dihydrochomeno [3,4-f] quinoline derivatives, and 6-thiophenehydroquinoline derivatives.

In certain embodiments, the progesterone agent is naturally occurring progesterone. In preferred embodiments, the progesterone agent is not a synthetic progesterone analog. In preferred embodiments, the progesterone agent is natural micronized progesterone.

Aspects of the subject methods include administering the progesterone agent to the subject as a monotherapy, e.g., as the only active agent, during the period of treatment. Accordingly, the subject is one who is not undergoing estrogen therapy.

Any convenient pharmaceutical compositions including a progesterone agent may be utilized according to the subject methods. The progesterone can be present in the compositions in any form known in the art. As needed, in the compositions of the present disclosure, the use of progesterone can be micronized, nano-sized, and/or amorphous forms. In some embodiments, the agent is micronized progesterone. FDA-approved bio-identical progesterone for hormone replacement therapy (HRT) is available as the branded stand-alone drug commercially identified as Prometrium® (Abbott Laboratories, Abbott Park, Ill.), and generic products provided by Teva (Israel) and Sofgen Americas, Inc (New York).

Conventional hormone replacement therapies utilize a high dose of progesterone or progesterone agent, such as a dose of 200 mg per day or more, 300 mg per day or more or even 400 mg per day or more. Treatment with low dosage progesterone agent according to the subject methods can improve many symptoms associated with menopause transition.

The terms “low therapeutically effective amount of a progesterone agent” and “low dosage progesterone agent” refer to a dosage regimen of progesterone agent administered to a subject that is 100 mg or less per day, such as 95 mg or less per day, 90 mg or less per day, 85 mg or less per day, 80 mg or less per day, 75 mg or less per day, 70 mg or less per day, 65 mg or less per day, 60 mg or less per day, 55 mg or less per day, 50 mg or less per day, 45 mg or less per day, 40 mg or less per day, 35 mg or less per day, 30 mg or less per day, 25 mg or less per day, or even less. In some instances of the subject methods, the daily dosage of the progesterone agent is from about 25 mg to about 75 mg. In some instances of the subject methods, the daily dosage of the progesterone agent is about 75 mg. In some instances of the subject methods, the daily dosage of the progesterone agent is about 50 mg. In some instances of the subject methods, the daily dosage of the progesterone agent is about 25 mg.

The low daily dosage of the progesterone agent can be administered via any convenient regimen. The pharmaceutical formulation can be administered in multiple doses per day, if desired, to achieve the total desired daily dose. In certain instances of the subject methods, the low therapeutically effective amount of the progesterone agent is administered via twice daily doses (bid). In some instances, administration may be once daily, such as each night at bedtime (qhs).

The subject dosage regimen can be performed for any convenient period of time, such as a period of one week or more, two weeks or more, three weeks or more, one month or more, 2 months or more, 3 months or more, 4 months or more, 5 months or more, 6 months or more, 7 months or more, 8 months or more, 9 months or more, 10 months or more, 11 months or more, 12 months or more, or even more, as needed. The subject dosage regimen can be performed for a period of about 1 week to about 4 weeks, such as about 1 week, about 2 weeks, about 3 weeks or about 1 month. In certain instances, the subject dosage regimen can be performed for a period of about 7 days to about 90 days, from about 7 days to about 60 days, from about 7 days to about 30 days, from about 7 days to about 21 days, or from about 7 days to about 14 days.

The progesterone agent can be administered via any convenient route of administration (e.g., oral, subcutaneous, intraperitoneal, intrauterine, sublingual, or intramuscular routes) by using standard methods. In addition, the pharmaceutical formulations can be administered to the woman via injectable depot routes of administration such as by using 1, 3, or 6-month depot injectable or biodegradable materials and methods. Typically, the woman will be treated over the course of a month, even several months or years, to ameliorate the signs and symptoms resulting from the menopause transition state.

In certain instances, the dosage regimen includes oral administration of low dose tablets of the progesterone agent via the regimen of two tablets qhs (each night at bedtime). In certain instances, the dosage regimen includes oral administration of low dose tablets of the progesterone agent via the regimen of one tablet qam (every morning) and one tablet qhs (each night at bedtime). In certain instances, the dosage regimen includes oral administration of low dose tablets of the progesterone agent via the regimen of one tablet qam (every morning) and two tablets qhs (each night at bedtime). In the dosage regimens described herein, the “low dose tablets of the progesterone agent” can refer to a tablet consisting of about 25 mg of the progesterone agent.

In some embodiments, the dosage form of progesterone agent is an oral dosage form. In some embodiments, the oral dosage form is controlled release. In one embodiment, the progesterone can be present or added to the oral dosage form as unmicronized, milled and sieved forms. In another embodiment, the oral dosage form can include a combination of these forms. The progesterone can be solubilized in one or more of the other components of the oral dosage form, such as the carrier, or it can be suspended within the oral dosage form. The suspended portion of progesterone may be partially or completely in unmicronized, milled, sieved, or amorphous forms or combinations thereof.

The progesterone agent can be partially or fully in the form of a high-energy solid which increases the dissolution rate in an aqueous medium significantly compared to at least one of its unmilled or unmicronized crystalline forms (low-energy forms). Examples of high-energy forms include amorphous forms and the like. In one embodiment the high-energy form progesterone of present disclosure may be physico-chemically pure. In yet another embodiment the high-energy form progesterone is physically and/or chemically associated with at least one additional substance, such as for example alcohol, pyrollidone, cellulose, polyol, polyethylene glycol, dextrins, cyclodextrins and the like. Several methods known in the art may be used to produce the high-energy form progesterone of the present disclosure, for example co-precipitation, solid-solution, co-melting, co-grinding, spray drying with co-solvent, controlled precipitation from super-saturated solutions, solidified super-saturated solutions, and combinations thereof.

Depending on the form of the progesterone, the compositions of the present disclosure could include dissolution-rate enhancers such as for example, wetting agents, surfactants, and the like. In one embodiment the compositions comprise at least one wetting agent and/or surfactant selected from the group comprising hydrophilic, lipophilic, amphiphilic, ionic, non-ionic surfactants. In another embodiment, the composition can be substantially free of added hydrophilic surfactants.

In some embodiments, the oral dosage form can include oils containing omega fatty acids. Non-limiting examples of oils containing omega fatty acids, can include, but are not limited to, a-linolenic acid (ALA), eicosapentaenoic acid (EPA), and docosahexaenoic acid (DHA), all of which are polyunsaturated with carbon chain length greater than 20. In another embodiment omega-3 fatty acids can be administered with progesterone agent concomitantly or sequentially.

The oral dosage forms can include a pharmaceutically acceptable carrier. The carrier can be a single ingredient, or a mixture of ingredients. Additionally, the carrier can take the form of an encapsulation coat, an absorbing agent, a coating substance, a controlled release device, a release modifying or release controlling agent, surfactants, or a combination thereof. When the carrier includes a surfactant, the surfactant may increase the solubility of the progesterone or other active agent in the system. In some aspects, the carrier can comprise about 1 wt % to about 99 wt % of the total system. In one embodiment, the carrier can comprise about 5 wt % to about 95 wt % of the total system or formulation. In another embodiment, the carrier can comprise about 20 wt % to about 80 wt %. In yet a further embodiment, the carrier can comprise about 30 wt % to about 60 wt %. In one embodiment, the carrier can be admixed with the progesterone. In another embodiment, the carrier can adsorb, entrap, or encapsulate at least a portion of the progesterone. In yet another embodiment, the carrier can act to solubilize the progesterone.

Solid formulations for oral administration can contain suitable carriers or excipients, such as corn starch, gelatin, lactose, liposomes, acacia, sucrose, microcrystalline cellulose, kaolin, mannitol, dicalcium phosphate, calcium carbonate, sodium chloride, or alginic acid. Disintegrators that can be used include, without limitation, micro-crystalline cellulose, corn starch, sodium starch glycolate and alginic acid. Tablet binders that may be used include acacia, methylcellulose, sodium carboxy ethylcellulose, polyvinylpyrrolidone (Povidone), hydroxypropyl methylcellulose, sucrose, starch, and ethylcellulose. Lubricants that may be used include magnesium stearates, stearic acid, silicone fluid, talc, waxes, oils, and colloidal silica.

Liquid formulations for oral or sublingual administration typically are prepared in water or other aqueous vehicles. The liquid formulations also can include solutions, emulsions, syrups, and elixirs containing, together with the active ingredients, wetting agents, sweeteners, and coloring and flavoring agents. Various liquid and powder formulations can be prepared by conventional methods for inhalation by the woman.

In another embodiment, the carrier and the progesterone may be present separate from each other, but within a unit dosage form. In another embodiment, the carrier and the progesterone may be present as separate unit dosage forms suitable for concomitant or non-concomitant oral administration.

It is understood that any of the dosage regimens described herein can be adapted for topical administration to achieve the desired effects. In some embodiments, the pharmaceutical formulations of the disclosure are contained within a transdermal patch. Numerous transdermal patches are known in the art and can readily be adapted to contain and deliver the pharmaceutical formulations of the disclosure. Examples of suitable transdermal patches are disclosed in U.S. Pat. Nos. 5,223,261; 3,598,123; 4,460,372; 3,598,122; 4,573,996; and 4,624,665. Typical transdermal patches have a flexible backing, a drug reservoir layer, a semipermeable membrane, and an adhesive layer coated on the exterior surface of the semipermeable membrane. Theratech patch technology, for example, can be used in the methods of the disclosure. If desired, the patch may contain a skin penetration enhancer (e.g., a fatty acid ester of a fatty acid such as ethyl oleate, glyceryl monolaurate, and/or isopropyl myristate).

In an alternative patch, the pharmaceutical formulation is contained within the adhesive coating, rather than in a distinct drug reservoir layer. Such a patch may contain, for example, a flexible backing (e.g., polyethylene, polypropylene, polyurethane, and the like) and a pressure-sensitive adhesive coating contiguously adhered to one surface of the backing and containing a homogenous mixture of: (i) an acrylic polymer containing a hydrophobic monomeric acrylic or methacrylic ester of an alkyl alcohol (containing 4-10 carbons), polyanhydrides, polyvinylacetate, polylactide or polyglycolide mixes; (ii) the active ingredients, each in an amount of about 0.2 to 12 percent of the total weight of the adhesive coating; and (iii) a skin penetration enhancer that includes isopropyl myristate and glyceryl monolaurate each in an amount of about 1 to 20 percent of the weight of the adhesive coating. These examples are non-limiting, and other transdermal patches can be used in conjunction with the methods of the disclosure.

Injectable formulations can contain various carriers such as vegetable oils, dimethylacetamide, dimethylformamide, ethyl lactate, ethyl carbonate, isopropyl myristate, ethanol, polylactide, polyglycolide, polyols, (glycerol, propylene glycol, liquid polyethylene glycol, and the like). For intravenous injections, the compound may be administered by the drip method, whereby a pharmaceutical formulation containing the active ingredient and a pharmaceutically acceptable carrier is infused. Pharmaceutically acceptable carries can include, for example, 5% dextrose, 0.9% saline, Ringer's solution or other suitable carriers. For intramuscular preparations, a sterile formulation containing the active ingredients can be administered in a pharmaceutical carrier such as Water-for-Injection, 0.9% saline, or 5% glucose solution.

A topical semi-solid ointment formulation typically contains a concentration of the active ingredients from about 1 to 20% (e.g., 5 to 10%) in a carrier such as a pharmaceutical cream base.

Various formulations for topical use include drops, tinctures, lotions, creams, solutions, and ointments containing the active ingredient and various supports and vehicles. The pharmaceutical formulations that find use in the methods of the disclosure can be administered to the woman via a variety of combinations of routes of administration.

### Kits

Kits including two or more doses of a progesterone agent (e.g., as described herein) are provided. In some cases, the kit includes multiple doses of the progesterone agent to provide for a low dosage regimen according to the subject methods (e.g., as described herein). In some cases, the kit includes two or more unit doses of the progesterone agent e.g., in an oral or transdermal dose form. In some embodiments, the kit includes two or more doses of 25 mg or less of a pharmaceutical composition including a progesterone agent as the only active agent.

In some embodiments of the kit, the progesterone agent is progesterone, such as micronized progesterone.

In the subject kits, the one or more components are present in the same or different containers, as may be convenient or desirable. In addition to the containers containing the components of the kits (e.g., unit doses) instructions can be included describing the use and attendant benefits of the progesterone agent in treating or preventing menopausal symptoms. Instructions may be provided in a variety of different formats. In certain embodiments, the instructions may include complete protocols for practicing the subject methods or means for obtaining the same (e.g., a website URL directing the user to a webpage which provides the instructions), where these instructions may be printed on a substrate, where substrate may be one or more of: a package insert, the packaging, reagent containers and the like. The instructions can be for administration of the progesterone agent to a subject according to a dosage regimen of 100 mg or less of the progesterone agent per day.

### Definitions

Before describing exemplary embodiments in greater detail, the following definitions are set forth to illustrate and define the meaning and scope of the terms used in the description.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

It must be noted that as used herein and in the appended claims, the singular forms “a”, “an”, and “the” include plural referents unless the context clearly dictates otherwise. For example, the term “a primer” refers to one or more primers, i.e., a single primer and multiple primers. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

An “estrogen” is an agent, natural or synthetic, that exerts biological effects characteristic of estrogenic hormones such as estradiol. The term “estrogen” also encompasses “conjugated estrogens,” which are an amorphous preparation of naturally occurring, water-soluble, conjugated forms of mixed estrogens that typically are obtained from the urine of pregnant mares (e.g., sodium estrone sulfate). Also included are “esterified estrogens,” which are a mixture of the sodium salts of sulfate esters or glucanoride of sulfate conjugates of estrogenic substances. Examples of estrogens which are excluded from certain embodiments of the subject methods include, without limitation, estradiol valerate, estradiol benzoate, 17-β estradiol, estradiol cypionate, estrone, piperazine estrone sulfate, estriol, ethyl estradiol, polyestradiol phosphate, estrone potassium sulfate, benzestrol, chlorotrianisene, methallenestril, dienestrol, diethylstilbestrol diphosphate, mestranol, DES, quinestranol, phytoestrogens, animal-derived estrogens (e.g., equine estrogens), and metabolic derivatives of animal-derived estrogens.

A “postmenopausal” woman is one who, in the absence of other medication, would experience at least 12 months of amenorrhea or levels of serum follicle-stimulating hormone greater than 30 mIU/mL.

By “treatment” is meant at least an amelioration of the symptoms associated with the pathological condition afflicting the subject, where amelioration is used in a broad sense to refer to at least a reduction in the magnitude of a parameter, e.g., symptom, associated with the pathological condition being treated, such as the degree of pain, or other associated side effects. The effect may be prophylactic in terms of completely or partially preventing a disease or symptom thereof and/or may be therapeutic in terms of a partial or complete cure for a disease and/or adverse effect attributable to the disease. The present method of “treating” a patient, as the term is used herein, thus encompasses both prevention of a disorder or symptom (e.g. pelvic pain) in a predisposed individual and treatment of the disorder or symptom in a clinically symptomatic individual.

By “therapeutically effective” or “effective amount” is meant a nontoxic but sufficient amount of an active agent (e.g. progesterone) given to a subject to provide the desired therapeutic effect. An effective amount will be a dosage sufficient for reduction or cessation of pelvic pain. The effective amount will vary with the age and physical condition of the subject, the severity of the pain being treated, the nature of any underlying condition being treated, the duration of the treatment, the nature of any concurrent treatment, the pharmaceutically acceptable carrier used if any, and analogous factors within the knowledge and expertise of those skilled in the art.

The term “unit dosage form” refers to physically discrete units suitable as unitary dosages for human and animal subjects, each unit containing a predetermined quantity of the subject compound of the present disclosure calculated in an amount sufficient to produce the desired effect in association with a pharmaceutically acceptable diluent, carrier or vehicle. The specifications for the unit dosage forms of the present disclosure depend on the particular compound employed and the effect to be achieved, and the pharmacodynamics associated with each compound in the host. Dose levels can vary as a function of the specific compound, the nature of the delivery vehicle, and the like. Desired dosages for a given compound are readily determinable by a variety of means.

The dose administered to an animal, particularly a human, in the context of the present disclosure should be sufficient to effect a prophylactic or therapeutic response in the subject over a reasonable time frame, e.g., as described in greater detail herein. Dosage will depend on a variety of factors including the strength of the particular compound or composition employed, the condition of the subject, and the body weight of the subject, as well as the severity of the symptoms and condition. The size of the dose will also be determined by the existence, nature, and extent of any adverse side-effects that might accompany the administration of a particular compound.

In some cases, the term “micronized progesterone,” as used herein, includes micronized progesterone having an X50 particle size value below about 15 microns and/or having an X90 particle size value below about 25 microns. In certain instances, the term micronized progesterone refers to Prometrium.

The term “X50,” as used herein, means that one-half of the particles in a sample are smaller in diameter than a given number. For example, micronized progesterone having an X50 of 5 microns means that, for a given sample of micronized progesterone, one-half of the particles have a diameter of less than 5 microns. Similarly, the term “X90” means that ninety percent (90%) of the particles in a sample are smaller in diameter than a given number.

Other definitions of terms may appear throughout the specification.

It is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

The section headings used herein are for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

The citation of any publication is for its disclosure prior to the filing date and should not be construed as an admission that the present claims are not entitled to antedate such publication by virtue of prior invention. Further, the dates of publication provided can be different from the actual publication dates which can be independently confirmed.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

All patents and publications, including all sequences disclosed within such patents and publications, referred to herein are expressly incorporated by reference.

### EXAMPLES

Below are examples of specific embodiments for carrying out the present invention. the examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### Example 1: Role of Low Dose Micronized Progesterone for the Management of Peri Menopausal Symptoms

Menopause is defined as the cessation of menses for 12 months. The median age of menopause in the United States is 51 years of age. The years leading up to, and shortly after menopause, is referred to as perimenopause or the “transition state”. Greater than 50% of women will experience symptoms during menopause transition that will impact their quality of life. Most women who have symptoms related to hormonal changes present in the this time period. The corpus luteum is a structure in the female ovary that occurs with ovulation. The corpus luteum is responsible for producing relatively high levels of progesterone. During the menopause transition time, ovulation becomes less frequent and often ceases. Lack of an adequate corpus luteum cyst results in a decline in progesterone production.

Many women who suffer from hot flashes, mood swings, breast tenderness, and sleep issues during this time period likely produce less progesterone in relationship to estrogen. The biosynthesis of estrogens differs between the premenopausal and postmenopausal woman. In the premenopausal woman, estrogen is primarily made in the ovary. In the postmenopausal women, estrogen is synthesized in peripheral sites. The main source of estrogen in the obese postmenopausal woman is the fat tissue. Fat tissue produces excess amounts of estrogen which can put a woman at risk of breast, endometrial, ovarian, and other cancers.

Not uncommonly, providers will prescribe the oral contraceptive pill or hormone replacement therapy to alleviate these symptoms. The oral contraceptive pill is significantly more potent than hormone replacement therapy. Hormone replacement therapy typically results in the administration of either estrogen alone, in the patient absent a uterus, or a combination of estrogen and progesterone in the patient with an intact uterus. The Women's Health Initiative findings noted an increased risk of cardiovascular disease, stroke, and breast cancer with hormone replacement therapy. As women transition into menopause, the production of estrogen and progesterone declines. Similar to polycystic ovarian syndrome, the menopause transition state for many women can lead to an estrogen dominant state. Estrogen dominance can result in breast tenderness, fatigue, sleep disturbances, hot flashes, worsening headaches, and emotional lability. It is widely accepted that estrogen dominance increases a woman's risk of cardiovascular disease, stroke, uterine, and breast cancer. This is seen in the obese patient who is at risk secondary to an increase in endogenous estrogen. Pregnancy and breast feeding, both progesterone high states, result in a decreased risk of breast and uterine cancer. Treating women with a low dose progesterone can alleviate many of these symptoms and negate the added risks of estrogen therapy. Current doses of progesterone typically prescribed are at a dose greater than most women need, resulting in side effects such as bloating or irritability.

It is apparent based on initial observations that supplementing with a low dose progesterone alleviates symptoms in women transitioning into menopause. The question then becomes, if a woman is experiencing symptoms of estrogen dominance does failing to treat estrogen dominance result in a greater risk of breast cancer, uterine cancer, and cardiovascular disease. Supplementing with progesterone, e.g., at 25 mg twice a day can improve many symptoms associated with menopause transition. Of the greater than 100 patients who underwent a trial of progesterone for treating symptoms of the menopause transition state, not one patient was subsequently diagnosed with breast cancer, uterine cancer, or a cardiovascular event.

### Exemplary Dosing Regimen Utilized for Treating Perimenopause Patients

Micronized progesterone (25 mg)
Dosing regimens of interest:

One tablet bid (twice a day);

Two tablets qhs (each night at bedtime);

One tablet qam (every morning), two tablets qhs (each night at bedtime).

### Example 2: Menopause Transition: An Observational Study on the Effectiveness of Low Dose Micronized Progesterone to Alleviate Symptoms

### Introduction

Menopause transition is the time period before and shortly after the cessation of the menstrual cycle. This time period results in a number of symptoms for many women to include; mood changes, hot flushes, breast tenderness, sleep problems, fatigue, and exhaustion. Common treatment options offered to women include; selective serotonin reuptake inhibitors, oral contraceptive pills, high dose progesterone, or hormone replacement therapy. These options often have side effects and may be ineffective. The purpose of this study was to determine the effectiveness of a low dose progesterone (e.g., micronized progesterone) in alleviating symptoms associated with menopause transition.

### Methods

Women between the ages of 40 to 55 years of age who were experiencing symptoms consistent with menopause transition were offered a trial of a low dose progesterone to alleviate their symptoms. The primary complaint for inclusion in the study was alterations in mood defined as depressive mood, irritability, or anxiety on the Menopause Rating Scale (MRS). A score of 2-4 on these mood changes was required to be included in the study. One additional symptom (sleep problems, hot flushes, physical and mental exhaustion) on the MRS was required, with a minimum score of 1, to be included. Patients with a history of breast cancer, actively trying to conceive, or in menopause were excluded from the study. Menopause was defined as no menses for greater than one year or surgical removal of the ovaries. Patients were consented for a trial of oral micronized progesterone at 25 mg twice a day. Response to treatment was evaluated in the office after 3 months of treatment. Data was collected over a one-year time period, September 2018-September 2019.

### Results

During the study period, 73 patients met the inclusion criteria. Four patients were lost to follow up. 14 patients opted to take micronized progesterone once a day in the morning or two tablets (50 mg) at night, and were excluded from the study. Of the 55 patients evaluated at 3 months, 52 patients (94.5%) were noted to have at least a two-point improvement in their MRS symptom score related to mood changes. 44 patients (80%) reported MRS symptom score of 0 at 3 months. Of the 32 patient who noted sleep problems, 27 patients (84%) had at least a one-point improvement in their MRS symptom score. Of the 48 patients who noted hot flushes, 44 patients (92%) had at least a one-point improvement in their MRS symptom score. Of the 19 patients who noted physical and mental exhaustion, 12 (63%) had at least a one-point improvement in their MRS symptom score. One patient discontinued the medication prior to the 3 month follow up secondary to worsening headaches. 54 patients did not report side effects to the medication. No patient discontinued the medication during the study period because of a diagnosis of breast cancer, thromboembolic event, or abnormal uterine bleeding. Of note, the patients who opted to alter the dosing regimen to once a day or two tablets at night noted benefits and chose to continue the medication.

### Discussion

The menopause transition time, referred to as perimenopause, includes both the early and late perimenopausal group. Patient selection was classified according to STRAW (Stages of Reproductive Aging Workshop) classification. Late perimenopause was defined as menstruation in the previous/last 2-12 months, but not in the previous/last 2 months. Early perimenopause was defined as increasing irregularity of menses without skipping periods (7 days difference) from the beginning of a given cycle to the next. The Menopause Rating Scale was utilized to quantify qualitative symptoms associated with the menopause transition. Given treatment differences in the symptoms that impact woman transitioning through menopause, attention was focused on 6 components of the MRS; hot flushes, sleep problems, depressive mood, irritability, anxiety, and physical/mental exhaustion. The scoring scheme increases point by point with increasing severity of subjectively perceived symptoms in each of those 6 items (severity 0 [no complaints] . . . 4 scoring points [very severe symptoms]). The respondent rated her personal symptoms on a 0 to 4 scale for each item. The MRS was chosen as a result of being a reliable measure of quality of life in aging women of various regions over time.

The corpus luteum is responsible for producing relatively high levels of progesterone. During the menopause transition, ovulation becomes less frequent and lack of an adequate corpus luteum cyst results in a decline of progesterone production. Many women who suffer from mood changes, hot flushes, sleep problems, and exhaustion during the menopause transition likely produce less progesterone relative to estrogen. Similar to polycystic ovarian syndrome, the menopause transition for many women can lead to an estrogen dominant state. This estrogen dominance can result in symptoms that significantly impact quality of life and place the patient at an increased risk of cardiovascular disease, stroke, and cancer of the uterus and breast.

Not uncommonly, providers will prescribe selective serotonin reuptake inhibitors, oral contraceptive pills, high doses of progesterone, and hormone replacement therapy to alleviate symptoms associated with the menopause transition state. Cascade et al. noted that thirty-eight percent of the approximately 700 patients surveyed reported experiencing one or more side effects as a result of taking an SSRI antidepressant. Of those patients (26%) indicated that these side effects were “very bothersome” or “extremely bothersome”. The oral contraceptive pill is significantly more potent than hormone replacement therapy. Hormone replacement therapy includes either the administration of estrogen alone, in the patient absent of a uterus, or a combination of estrogen and progesterone in the patient with an intact uterus. The Women's Health Initiative findings noted an increased risk of cardiovascular disease, stroke, and breast cancer with hormone replacement therapy. Furthermore, the progesterone administered to the participants was medroxyprogesterone acetate (MPA). Higher doses of micronized progesterone (100 mg and 200 mg) are commercially available, but often result in side effects. These may include bloating, nausea breast tenderness, headaches, drowsiness, and mood alterations in the woman transitioning into menopause.

The symptoms associated with the menopause transition state vary from patient to patient. These symptoms can often occur in a stable hormonal environment. It is often difficult to discern if symptoms are a result of a hormonal imbalance or related to other causes. For example, patients may have mood alterations due to a neurochemical imbalance or social stressors. Patients may experience hot flushes as the approach the later stages of late perimenopause due to lack of estrogen. Sleep disturbances may be related to sleep apnea. A secondary aim of this study was to determine what symptoms best identify those patients who would likely benefit from low dose micronized progesterone. Consistent, positive results were seen in the perimenopause patient with mood changes and one or more common symptoms found in the menopause transition time period. Although mood changes were the primary complaint of the participants, the majority of participants noted improvement in other symptoms associated with menopause transition. Symptoms of hot flushes, sleep problems, and physical/mental exhaustion were also alleviated with a low dose micronized progesterone. The Menopause Rating Scale is a cost effective and useful tool to determine which patients would likely benefit from treatment with low dose micronized progesterone.

Estrogen dominance is seen in the obese patient. These patients are at an increased risk of cardiovascular disease, uterine, and breast cancer secondary to an increase in endogenous estrogen. Although the criteria for inclusion in this study were based on clinical symptoms, laboratory data in the majority of the patients revealed estradiol levels in the premenopausal range while progesterone levels were often in the menopausal range. A woman transitioning into menopause is likely producing more estrogen relative to progesterone than during the premenopausal time period. Progesterone, specifically micronized progesterone, is showing promise as being protective for certain disease processes. The American Association of Clinical Endocrinologists and the American College of Endocrinology recommends micronized progesterone as a “safer option”. In addition, a decreased risk of histologic and hormone receptor—defined invasive breast cancer was noted with use of a combination of micronized progesterone and estrogen versus the use of synthetic progesterone.

For the purposes of this study, the menopausal patient was excluded, but that patient could be included in further studies using a low dose progesterone for menopausal symptoms. The question then becomes, if the woman transitioning into menopause is suffering from symptoms of estrogen dominance, does failing to treat those symptoms with a low dose progesterone put that patient at a greater risk of breast cancer, uterine cancer, and cardiovascular disease later in life. Further long-term studies are needed to answer that question.

### CONCLUSION

It is apparent based on initial results that treatment with a low dose progesterone alleviates symptoms in many women transitioning into menopause. Progesterone 25 mg twice a day can improve the quality of life for the woman in the early and late phases of menopause without side effects and no risks to date. In addition, progesterone at 25 mg allows flexibility of dosing once a day, twice a day, or two tablets at night depending on patient response to treatment.

### REFERENCES

### 1. Harlow, S. Margery, G. Hall, J. Lobo, R. Maki, P. Reber, R. Sherman, S. Sluss, P. Villiers, T. Executive Summary of the Stages of Reproductive Aging Workshop+10. Menopause. 2012 April, 19(4): 387-395

2. Heinemann L A J, Potthoff P, Schneider H P. International version of the menopause rating scale (MRS) Health Qual Life Outcomes 2003. p. 28.

### 3. Fertility and Sterility Vol. 76, No. 5, November 2001.

### 4. Cascade E, Kalali A, Kennedy S. Psychiatry. 2009 February; 6(2): 16-18.

5. Stefanick, M. Cochrone, B. Hsia, J. Barad, D. Liu, J. Johnson, S. The Women's Health Initiative Postmenopausal Hormone Trials: Overview of Participants. Ann Epidemiol 2003; 13: S78-S86.
6. Cobin, R. Goodman, N. Menopause Update. Endocrine Practice, Vol 23, No. 7, July 2017.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the embodiments shown and described herein.

1. A method for treating one or more symptoms of menopause in a subject, comprising:
administering to a subject who would benefit from such treatment a low therapeutically effective amount of a progesterone agent to treat the subject for the one or more symptoms of menopause.

2. The method of claim 1, wherein the low therapeutically effective amount of the progesterone agent is 100 mg or less per day.

3.-4. (canceled)

5. The method of claim 4, wherein the low therapeutically effective amount of the progesterone agent is 50 mg or less per day.

6.-7. (canceled)

8. The method of claim 7, wherein the low therapeutically effective amount of the progesterone agent is 25 mg per day.

9. The method of claim 1, wherein the progesterone agent is administered with doses of 25 mg.

10. The method of claim 1, wherein the progesterone agent is administered once per day.

11. (canceled)

12. The method of claim 1, wherein the progesterone agent is administered twice per day.

13. (canceled)

14. The method of claim 1, wherein the progesterone agent is administered three times or more per day.

15. The method of claim 1, wherein the progesterone agent is administered daily for a treatment period of two weeks or more.

16.-18. (canceled)

19. The method of claim 1, wherein the progesterone agent is progesterone.

20. The method of claim 19, wherein the progesterone is natural micronized progesterone.

21. The method of claim 1, wherein the method is a monotherapy.

22. The method of claim 21, wherein the progesterone agent is the only hormone replacement therapeutic agent administered to the subject.

23. The method of claim 1, wherein the progesterone agent is administered orally.

24.-26. (cancelled)

27. The method of claim 1, wherein the method at least ameliorates the one or more symptoms of menopause.

28. The method of claim 27, wherein the one or more symptoms of menopause is selected from alteration in mood, hot flashes/flushes, sleep disturbances, fatigue, physical and/or mental exhaustion, breast tenderness and emotional lability.

29. The method of claim 27, wherein the method results in a reduction in alteration of mood symptoms as measured on the Menopause Rating Scale (MRS).

30.-31. (canceled)

32. The method of claim 1, wherein the subject is a perimenopausal female.

33. A method of hormone replacement therapy in a subject, comprising:
administering to a perimenopausal woman a low therapeutically effective amount of progesterone as a monotherapy to treat the subject for progesterone deficiency.

34.-35. (canceled)

36. A kit for use in hormone replacement monotherapy, comprising:
two or more doses of 50 mg or less of a pharmaceutical composition comprising a progesterone agent as the only active agent;
instructions for administration of the progesterone agent to a subject according to a dosage regimen of 100 mg or less of the progesterone agent per day.

37.-39. (canceled)
